(19) United States
(12) Patent Application Publication
(10) Pub. No.: US 2019/0110484 A1
(43) Pub. Date: Apr. 18, 2019

(54) COMPOSITIONS FOR BAKED PRODUCTS CONTAINING LIPOLYTIC ENZYMES AND USES THEREOF

(71) Applicant: PURATOS NV, Groot-Bijgaarden (BE)

(72) Inventors: Jacques GEORIS, Liège (BE); Valérie DORGEO, Bertrix (BE); Oksana SHEGAY, Hasselt (BE); Thierry DAUVRIN, Couthuin (BE); Evelien AGACHE, Drongen (BE); Agnès DUTRON, Bruxelles (BE); Filip ARNAUT, Roosdaal (BE)

(21) Appl. No.: 16/091,052

(22) PCT Filed: Apr. 28, 2017
(86) PCT No.: PCT/EP2017/060144

§ 371 Date: Oct. 3, 2018

(30) Foreign Application Priority Data
Apr. 29, 2016 (BE) 2016/5307
May 18, 2016 (BE) 2016/5346

(51) Int. Cl. A21D 8/04; A21D 2/22; A21D 2/26; A23L 7/104; C12N 9/20

(52) U.S. Cl. CPC A21D 8/042; A21D 2/22; A21D 2/267; C12Y 301/01032; C12N 9/20; C12Y 301/01004; A23L 7/104

(57) ABSTRACT

The present invention relates to compositions and methods to improve properties of baked products that combine lipolytic enzymes having defined ratios of phospholipase A1 activity to phospholipase A2 activity.

### FIELD OF THE INVENTION

The present invention relates to novel compositions and methods for the improvement of bakery products that comprises one or more lipolytic enzymes.

### BACKGROUND OF THE INVENTION

In baking, flour lipids, though representing 2% of flour mass, play an important technological role because they interact with proteins and starch in a dough or a batter, influencing the rheological properties of the dough or the batter, as well as the baked product quality. The lipids can be divided into free lipids and bound lipids, both fractions containing either polar or nonpolar lipids. Approximately, half of the lipids are polar, and the ratio of polar to nonpolar lipids is of great importance in bread making because of its strong correlation with bread volume. The polar lipid fraction is mainly composed of lysophospholipids, phospholipids and galactolipids.

It is important to note that the lipids are located in different places in a dough or a batter. Most of the polar lipids are inside the starch granules. Some free lipids will spontaneously migrate to water gas interfaces, some are on the surface or inside starch granules or attached to gluten molecules and some will only be available after starch gelatinisation.

In general, the major function of lipids is their effect on gas cells stability and gluten strengthening. Also in particular the polar lipids have the ability to reduce starch retrogradation. Gas bubbles stabilization from yeast fermentation leads to larger baked product volume. Strengthening the gluten network leads to a better dough stability and enhances the crumb softness and texture therefore extending the shelf life. However, due to the minor amount of lipids in flour, the native phospholipid fraction of the flour is not enough to give a significant effect by itself on the properties of dough or batter and the quality of baked products. Moreover some of the phospholipid molecules present have no positive effect on dough properties or even a negative effect. Therefore, exogenous phospholipids and/or emulsifiers are used to ensure uniform quality and shelf life stability of baked products. Another way to address this issue is to use lipolytic enzymes (such as phospholipases and lipases) and do an in-situ modification of triglycerides, phospholipids and galactolipids to release the corresponding lysolipids. Lysolipids have better emulsifying properties compared to the original molecules and are more functional as wetting agent in bread and cake making processes.

Furthermore the release of more lysolipids having superior emulsifying properties leads to improved dough or batter rheological properties. Particularly, in cake making, such improved release enhances the air incorporation in the aqueous phase in foam or sponge cake and improves the dispersion of the bakery fat in the batter of layer cakes.

Due to the fact that the lipids are not evenly distributed in dough and batter systems they are not always accessible for hydrolysis by lipases and phospholipases. By changing the pH conditions, the ionic strength and/or the sugar concentration at different temperatures the availability of these lipids will be modified. In such cases one needs enzymes that are active at these different temperatures and conditions and have the desired specificity. On the other hand, hydrolysing the substrates too far will gives rise to molecules with negative effect on baked products quality characteristics.

Although some lipases and/or phospholipases have already been described for their positive properties in the preparation of baked products, the outcome of their use is highly unpredictable, due to their different specificities, their different hydrolysis products, their potential synergies or the process conditions or substrates. Therefore, today, there is still a need for compositions and methods to further improve properties of baked products such as dough or batter tolerance, volume and/or freshness.

### SUMMARY OF THE INVENTION

The inventors have found that the use of combination of a lipolytic enzyme and a particular phospholipase in bakery applications, and in particular in bread making, has a synergistic effect on dough tolerance.

Accordingly, in a first aspect, the present invention relates to a composition comprising:
at least one first enzyme having a phospholipase A1 activity/phospholipase A2 activity ratio ranging between 0.01 and 100, preferably ranging between 0.01 and 80, more preferably ranging between 0.01 and 50, even more preferably ranging between 1 and 50, even more preferably ranging between 2 and 50, even more preferably ranging between 0.01 and 25, even more preferably ranging between 1 and 25, and even more preferably ranging between 2 and 25; and;
at least one second enzyme having a phospholipase A1 activity/phospholipase A2 activity ratio ranging between 5000 and 60000, preferably between 10000 and 50000, more preferably between 15000 and 50000; and a phospholipase A1 activity/lipase activity ratio of 500 or more.

In a particular embodiment the composition as disclosed herein provides that said first enzyme is chosen from a lipolytic enzyme with phospholipase activity from Chaetomium thermophilum, a lipolytic enzyme with phospholipase activity from Meiothermus ruber, a lipolytic enzyme with phospholipase activity from Meiothermus silvanus, a lipolytic enzyme with phospholipase activity from Streptomyces violaceoruber and/or a lipolytic enzyme with phospholipase activity from Fusarium culmorum, preferably a lipolytic enzyme with phospholipase activity from Meiothermus ruber and/or a lipolytic enzyme with phospholipase activity from Meiothermus silvanus, more preferably a lipolytic enzyme with phospholipase activity from Meiothermus silvanus.

In a particular embodiment the composition as disclosed herein provides that said first enzyme has a sequence identity of at least 85% with any of SEQ ID NO 1, SEQ ID NO 2, SEQ ID NO 3, SEQ ID NO 4 and/or SEQ ID NO 5, preferably having a sequence identity of at least 85% with any of SEQ ID NO 1, SEQ ID NO 2 and/or SEQ ID NO 3, more preferably having a sequence identity of at least 85% with SEQ ID NO 2 and/or SEQ ID NO 3, and even more preferably having a sequence identity of at least 85% with SEQ ID NO 3.

In a particular embodiment the composition as disclosed herein provides that said first enzyme is characterized by having an optimum phospholipase activity at a temperature equal or higher than 45° C.

In a particular embodiment the composition as disclosed herein provides that said first enzyme retains more than 50% of its phospholipase activity after being incubated for 30 minutes at 50° C.

In a particular embodiment the composition as disclosed herein provides that said second enzyme is chosen from a lipolytic enzyme with phospholipase and lipase activities from Thermomyces lanuginosus or a lipolytic enzyme with phospholipase and lipase activities from Fusarium solani.

In a particular embodiment the composition as disclosed herein provides that said second enzyme has a sequence identity of at least 85% with any of SEQ ID NO 6 and/or SEQ ID NO 7 or is chosen from Lipopan® Max from Novozymes or Veron® Hyperbake T from AB enzymes.

Furthermore, in a further aspect, the present invention relates to the use of a composition as disclosed herein in bakery applications.

In a particular embodiment the use of the composition as disclosed herein in bread improvers is provided.

In a particular embodiment the use of the composition as disclosed herein in bread or patisserie products, preferably cakes, bread, baguettes or rolls is provided.

Furthermore, in a further aspect, the present invention relates to a bread improver comprising the composition as disclosed herein.

Furthermore, in a further aspect, the present invention relates to a method for preparing a baked product, comprising the steps of adding to the dough or batter, prior to baking:
at least one first enzyme having a phospholipase A1 activity/phospholipase A2 activity ratio ranging between 0.01 and 100, preferably ranging between 0.01 and 80, more preferably ranging between 0.01 and 50, even more preferably ranging between 1 and 50, even more preferably ranging between 2 and 50, even more preferably ranging between 0.01 and 25, even more preferably ranging between 1 and 25, and even more preferably ranging between 2 and 25; and;
at least one second enzyme having a phospholipase A1 activity/phospholipase A2 activity ratio ranging between 5000 and 60000, preferably between 10000 and 50000, more preferably between 15000 and 50000; and a phospholipase A1 activity/lipase activity ratio of 500 or more.

In a particular embodiment the method as disclosed herein provides that said dough or batter comprises between 5000 and 100000 PmU/100 kg flour, preferably between 7000 and 50000 PmU/100 kg flour, more preferably between 10000 and 30000 PmU/100 kg flour of said first enzyme and between 10000 and 100000 LmU/100 kg flour, preferably between 20000 and 70000 LmU/100 kg flour of said second enzyme.

In a particular embodiment the method as disclosed herein provides that said dough or batter shows improved tolerance.

In a particular embodiment the method as disclosed herein provides that said baked product shows improved freshness.

Furthermore, in a further aspect, the present invention relates to a baked product prepared from a dough or batter comprising the composition as disclosed herein.

### DETAILED DESCRIPTION

Before the present products, compositions, uses and methods of the invention are described, it is to be understood that this invention is not limited to particular products, compositions, uses and methods or combinations described, since such products, compositions, uses and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any or etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Disclosed herein are compositions comprising a first enzyme having a low phospholipase A1 activity/phospholipase A2 activity ratio combined with a second enzyme having a high phospholipase A1 activity/phospholipase A2 activity ratio which is in particular suitable for use in bakery applications and in particular for use as or in a bread improver.

The inventors have surprisingly found that the use of compositions that comprises two or more lipases and/or phospholipases with the particular properties as disclosed herein allows obtaining baked products with improved characteristics. In particular the properties (e.g. tolerance) of the batter and/or dough are improved considerably. Also the baked products have been found to show improved volume and/or freshness.

As used herein, the term “lipase” refers generally to triacylglycerol lipases or triacylglycerol acylhydrolase as defined by enzyme entry EC 3.1.1.3. Lipases are defined herein as enzymes that catalyze the hydrolysis of triacylglycerols to give free fatty acids, diacylglycerols, monoacylglycerols and glycerol. The lipase used in the compositions defined herein may comprise enzymatic side-activities such as for example phospholipase activity.

In the context of the present invention the lipase activity is measured using p-nitrophenyl palmitate (pNPP) as substrate and according to the method described herein. The enzyme activity can also be measured with other assays for lipase activity known by persons skilled in the art (for a review see for example Stoytcheva M. & al, 2012, Current Analytical Chemistry, vol 8, p. 400).

In particular, the lipase activity is measured using p-nitrophenyl palmitate (pNPP) as substrate. The release of yellow p-nitrophenol due to hydrolysis of p-nitrophenyl palmitate by lipase is measured by spectrophotometry at 414 nm. One lipase milliunit (LmU) is defined as the amount of enzyme needed to release one nanomole (nmole) per minute of p-nitrophenol from p-nitrophenyl palmitate at 40° C. and pH 7.5. More details on the lipase activity measurement are given in the examples.

As used herein, the term “phospholipase” refers generally to enzymes that hydrolyse phospholipids into fatty acids and other lipophilic substances like for example lysophospholipids, diacylglycerols, choline phosphate and phosphatidates, depending on the site of hydrolysis. Depending on the specific bond targeted in the phospholipids molecule, phospholipases are classified in different types such as A, B, C and D.

In the context of the present invention the phospholipase activity is measured using p-nitrophenyl phosphorylcholine as substrate, wherein the phospholipase activity is expressed in milliunits (PmU) that is defined as the amount of enzyme needed to release one nanomole (nmole) of p-nitrophenol from p-nitrophenyl phosphorylcholine per minute at 50° C. and pH 7.5. The phospholipase activity can also be measured with other assays for phospholipase activity known by persons skilled in the art such as the hydrolysis of phosphatidylcholine.

In particular, the phospholipase activity is measured using p-nitrophenyl phosphorylcholine (pNPPC) as substrate. The release of yellow p-nitrophenol due to hydrolysis of p-nitrophenyl phosphorylcholine by phospholipase is measured by spectrophotometry at 414 nm. One phospholipase milliunit (PmU) is defined as the amount of enzyme needed to release 1 nmol of p-nitrophenol per minute at 50° C. and pH7.5. More details on the phospholipase activity measurement are given in the examples.

As used herein, the term “phospholipase A” refers to lipolytic enzymes that catalyse the hydrolysis of one or more bonds in phospholipids. Two different types of phospholipase A activity can be distinguished which hydrolyse the ester bond(s) that link the fatty acyl moieties to the glycerol backbone. Phospholipase A1, as defined by enzyme entry EC 3.1.1.32, and Phospholipase A2, as defined by enzyme entry EC 3.1.1.4, catalyse the deacylation of one fatty acyl group in the sn-1 and sn-2 positions respectively, from a diacylglycerophospholipid to produce lysophospholipid.

In the context of the present invention the phospholipases activities are measured using respectively a lipid mix of dioleoylphosphatidylcholine, dioleoylphosphatidylglycerol and dye labelled N-((6-(2,4-DNP)Amino)Hexanoyl)-1-(BODIPY® FL C5)-2-Hexyl-Sn-Glycero-3-Phosphoethanolamine (for phospholipase A1) or a lipid mix of dioleoylphosphatidylcholine, dioleoylphosphatidylglycerol and dye labelled 1-O-(6-BODIPY® 558/568-Aminohexyl)-2-BODIPY® FL C5-Sn-Glycero-3-Phosphocholine (for phospholipase A2) as substrates and according to the methods described herein. The phospholipase activities can also be measured with other assays for lipase activity known by persons skilled in the art, such as using rac-1,2-S,O-didecanoyl-3-phosphocholine-1-mercapto-2,3-propanediol substrate for assaying phospholipase A1 activity and 2-hexadecanoylthio-1-ethyl-phosphocholine substrate for assaying phospholipase A2 activity.

In particular, the phospholipase A1 (PLA1) activity is measured using a lipid mix of dioleoylphosphatidylcholine, dioleoylphosphatidylglycerol and dye labelled N-((6-(2,4-DNP)Amino)Hexanoyl)-1-(BODIPY® FL C5)-2-Hexyl-Sn-Glycero-3-Phosphoethanolamine (PED-A1) as substrate (that can be found for example in the EnzChek Phospholipase A1 assay kit—ThermoFisher Scientific). The PED-A1 is specific for PLA1 and is a dye-labelled glycerophosphoethanolamine with BODIPY® FL dye-labelled acyl chain at the sn-1 position and dinitrophenyl quencher-modified head group. One phospholipase A1 milliunit (PA1mU) is defined as the amount of enzyme needed to release one nanomole (nmole) per minute of fluorescent fatty acid substituted at the sn-1 position of PED-A1 at 40° C. and pH7.4. More details on the phospholipase A1 activity measurement are given in the examples.

In particular, the phospholipase A2 (PLA2) activity is measured using a lipid mix of dioleoylphosphatidylcholine, dioleoylphosphatidylglycerol and dye labelled 1-O-(6-BODIPY® 558/568-Aminohexyl)-2-BODIPY® FL C5-Sn-Glycero-3-Phosphocholine (Red/Green BODIPY® PC-A2) as substrate (that can be found for example in the EnzChek Phospholipase A2 assay kit—ThermoFisher Scientific). The Red/Green BODIPY® PC-A2 substrate is selective for PLA2 and provides sensitive and continuous rapid real-time monitoring of PLA2 enzyme activities. One phospholipase A2 milliunit (PA2mU) is defined as the amount of enzyme needed to release one nanomole (nmole) per minute of fluorescent fatty acid substituted at the sn-2 position of Red/Green BODIPY® PC-A2 at 40° C. and pH 8.9. More details on the phospholipase activity A2 measurement are given in the examples.

Accordingly, in a first aspect, the present invention relates to a composition comprising:
at least one first enzyme having a phospholipase A1 activity/phospholipase A2 activity ratio ranging between 0.01 and 100, preferably ranging between 0.01 and 80, more preferably ranging between 0.01 and 50, even more preferably ranging between 1 and 50, even more preferably ranging between 2 and 50, even more preferably ranging between 0.01 and 25, even more preferably ranging between 1 and 25, and even more preferably ranging between 2 and 25; and;
at least one second enzyme having a phospholipase A1 activity/phospholipase A2 activity ratio ranging between 5000 and 60000, preferably between 10000 and 50000, more preferably between 15000 and 50000; and a phospholipase A1 activity/lipase activity ratio of 500 or more.

In particular the lipase activity is expressed in milliunits (LmU) that is defined as the amount of enzyme needed to release one nanomole (nmole) per minute of p-nitrophenol from p-nitrophenyl palmitate at 40° C. and pH 7.5, the phospholipase A1 activity is expressed in milliunits (PA1mU) that is defined as the amount of enzyme that hydrolyses one nanomole (nmole) per minute of fluorescent fatty acid substituted at the sn-1 position of N-((6-(2,4-DNP)Amino)Hexanoyl)-1-(BODIPY® FL C5)-2-Hexyl-Sn-Glycero-3-Phosphoethanolamine at 40° C. and pH7.4 and the phospholipase A2 activity is expressed in milliunits (PA2mU) that is defined as the amount of enzyme that hydrolyses one nanomole (nmole) per minute of fluorescent fatty acid substituted at the sn-2 position of 1-O-(6-BODIPY® 558/568-Aminohexyl)-2-BODIPY® FL C5-Sn-Glycero-3-Phosphocholine at 40° C. and pH 8.9.

In a particular embodiment the composition as disclosed herein comprises:
at least one first enzyme having a phospholipase A1 activity/phospholipase A2 activity ratio ranging between 0.01 and 50; and;
at least one second enzyme having a phospholipase A1 activity/phospholipase A2 activity ratio ranging between 15000 and 50000; and a phospholipase A1 activity/lipase activity ratio of 500 or more.

In a particular embodiment the composition as disclosed herein comprises:
at least one first enzyme having a phospholipase A1 activity/phospholipase A2 activity ratio ranging between 1 and 50; and;
at least one second enzyme having a phospholipase A1 activity/phospholipase A2 activity ratio ranging between 15000 and 50000; and a phospholipase A1 activity/lipase activity ratio of 500 or more.

In a particular embodiment the composition as disclosed herein comprises:
at least one first enzyme having a phospholipase A1 activity/phospholipase A2 activity ratio ranging between 2 and 50; and;
at least one second enzyme having a phospholipase A1 activity/phospholipase A2 activity ratio ranging between 15000 and 50000; and a phospholipase A1 activity/lipase activity ratio of 500 or more.

In a particular embodiment the composition as disclosed herein comprises:
at least one first enzyme having a phospholipase A1 activity/phospholipase A2 activity ratio ranging between 0.01 and 25; and;
at least one second enzyme having a phospholipase A1 activity/phospholipase A2 activity ratio ranging between 15000 and 50000; and a phospholipase A1 activity/lipase activity ratio of 500 or more.

In a particular embodiment the composition as disclosed herein comprises:
at least one first enzyme having a phospholipase A1 activity/phospholipase A2 activity ratio ranging between 1 and 25; and;
at least one second enzyme having a phospholipase A1 activity/phospholipase A2 activity ratio ranging between 15000 and 50000; and a phospholipase A1 activity/lipase activity ratio of 500 or more.

In a particular embodiment the composition as disclosed herein comprises:
at least one first enzyme having a phospholipase A1 activity/phospholipase A2 activity ratio ranging between 2 and 25; and;
at least one second enzyme having a phospholipase A1 activity/phospholipase A2 activity ratio ranging between 15000 and 50000; and a phospholipase A1 activity/lipase activity ratio of 500 or more.

The inventors have found that the enzymes of the compositions as disclosed herein act synergistically in the improvement of the baked product properties.

In a particular embodiment the composition as disclosed herein provides that said first enzyme is characterized by having an optimum phospholipase activity at a temperature equal or higher than 45° C.

In a particular embodiment the composition as disclosed herein provides that said first enzyme is chosen from a lipolytic enzyme with phospholipase activity from Chaetomium thermophilum, a lipolytic enzyme with phospholipase activity from Meiothermus ruber, a lipolytic enzyme with phospholipase activity from Meiothermus silvanus, a lipolytic enzyme with phospholipase activity from Streptomyces violaceoruber and/or a lipolytic enzyme with phospholipase activity from Fusarium culmorum, preferably a lipolytic enzyme with phospholipase activity from Meiothermus ruber and/or a lipolytic enzyme with phospholipase activity from Meiothermus silvanus, more preferably a lipolytic enzyme with phospholipase activity from Meiothermus silvanus.

In a particular embodiment the composition as disclosed herein provides that said first enzyme has a sequence identity of at least 85% with any of SEQ ID NO 1, SEQ ID NO 2, SEQ ID NO 3, SEQ ID NO 4 and/or SEQ ID NO 5 (see table A), and preferably having a sequence identity of at least 85% with any of SEQ ID NO 1, SEQ ID NO 2 and/or SEQ ID NO 3, more preferably having a sequence identity of at least 85% with SEQ ID NO 2 and/or SEQ ID NO 3, and even more preferably having a sequence identity of at least 85% with SEQ ID NO 3.

More particular said first enzyme is a lipolytic enzyme having a sequence identity of at least 85%, preferably at least 90%, more preferably at least 95% to SEQ ID NO 1, SEQ ID NO 2, SEQ ID NO 3, SEQ ID NO 4 and/or SEQ ID NO 5, and preferably having a sequence identity of at least 85%, preferably at least 90%, more preferably at least 95% with any of SEQ ID NO 1, SEQ ID NO 2 and/or SEQ ID NO 3, more preferably having a sequence identity of at least 85%, preferably at least 90%, more preferably at least 95%, with SEQ ID NO 2 and/or SEQ ID NO 3, and even more preferably having a sequence identity of at least 85%, preferably at least 90%, more preferably at least 95% with SEQ ID NO 3 (see Table A).

TABLE A

| Name | Sequence |
|---|---|
| SEQ ID NO 1 | MKGFLLASLAALAVAAPSSKKQRAAPVTA |
| (protein) PH2 | QQLNNFKLYMQWSSASHCANEAPIGSVVT |
| [Chaetomium | CTDNQCSMFQSHNATVAATFIGSILDMRG |
| thermophilum] | FLGIDDVDKNIVLSFRGSTSWRNWIADAI |
| | FVQTPCDLTPGCLVHAGFYASWLEIKNSV |
| | IDAVKAAKAAHPNYKLVTTGHSLGAAVAT |
| | LAAATLRKAGIPIELYTYGSPRVGNKAFA |
| | EFVTNQAGGEYRLTHSADPIPRLPPIIFN |
| | YRHTSPEYWFDEGEDGVVTVDEFQICEGY |
| | ANVNCNAATSGFNMDLHGWYFQNHQGCSL |
| | GYTPWRAVKERELSDPELEALVNRFAEMD |
| | KAYVENLNLEGLEP |
| SEQ ID NO 2 | MGKPMRFAVGILALLLAACSQPATESTSA |
| (protein) PH3 | SIVEDMRQLGVEPEAIAAYTEALQNLEQA |
| [Meiothermus | RLALQSLGAGDLLLVQSIALGSVANYDAY |
| ruber] | YNARASYPQFDWSRNGCSAPEGLGLGYRE |
| | TFRPACNVHDFGYANFPRFPSLYNETGRK |
| | LSDDNFLVNMNQICRPRSFLSRSACYSAA |
| | YAYYLAVRSAGWAYFYD |
| SEQ ID NO 3 | MRVVLLMLVLVLAACGSQTGAPNWTLEQI |
| (protein) PH4 | AALTPEQIDALSEGELQKIQTILQPEFAR |
| [Meiothermus | VESQLIQLQSQLEALVAAQDSRWPNFDYT |
| silvanus] | VYLATAIPYGTFFTYYRTYSGPDWSNDGC |
| | SYSPDKPLFLNFKDPCNHHDFGYR |
| SEQ ID NO 4 | MRTTTRTRTTLAAVGAALALGVAAAPPQA |
| (protein) | APADKPQVLASFTQTSASSQNAWLAANRN |
| lipolytic | QSAWAAYEFDWSTDLCSQAPDNPFGFPFN |
| enzyme | TACARHDFGYRNYKAAGSFDANKSRIDSA |
| [Streptomyces | FYEDMKRVCTGYTGEKNTACNSTAWTYYQ |
| violaceoruber] | AVKIL |
| SEQ ID NO 5 | MVLDRLLFLLSLWLGFVGATQAALSEPIP |
| (protein) | PSKDPWYTAPPGFENAEPGTVFRVRPAPG |
| lipolytic | NLTSVIGNCSASYNILYRTTDSHFKPTWA |
| enzyme | VTTLLVPKLGPESLAQQKYQQSALLSFQV |
| [Fusarium | PYDSPDVDASPSNAMYDASDFFSNYYGAA |
| culmorum] | LGEGIFVSVPDYEGPLAAFTAGLISGYAT |
| | LDSIRAILSLGLGFNTIDTPSVALWGYSG |
| | GAFATEWASELAVQYAPELVAGPVIGAVM |
| | GAPLPNITSCMRDVNGGPKSGLVVNILLG |
| | LTGQYPDVRKHLVSKLNDDGQYNKAGFLA |
| | AEGFTISEALSTFSGNINKYFQKGTDILS |
| | DPKITALINREGVLGYHGTPRWPMFIYQA |
| | ISDEVTPIAATDAVVERYCSVGADVHFER |
| | NTLGSHDEEAGNSYDAAFQWLLDIFSGQR |
| | DTKGCVIKDVTRDVTGDVTGDVTRDVTRE |
| | L |
| SEQ ID NO 6 | MRSSLVLFFVSAWTALASPIRREVSQDLF |
| (protein) | NQFNLFAQYSAAAYCGKNNDAPAGTNITC |
| lipolytic | TGNACPEVEKADATFLYSFEDSGVGDVTG |
| enzyme | FLALDNTNKLIVLSFRGSRSIENWIGNLN |
| [Thermomyces | FDLKEINDICSGCRGHDGFTSSWRSVADT |
| lanuginosus] | LRQKVEDAVREHPDYRVVFTGHSLGGALA |
| | TVAGADLRGNGYDIDVFSYGAPRVGNRAF |
| | AEFLTVQTGGTLYRITHTNDIVPRLPPRE |
| | FGYSHSSPEYWIKSGTLVPVTRNDIVKIE |
| | GIDATGGNNQPNIPDIPAHLWYFGLIGTC |
| | L |
| SEQ ID NO 7 | MMLILSILSIIAFTAAGPVPSVDENTRVL |
| (protein) | EHRAVTVTTQDLSNFRFYLQHADAAYCNF |
| lipolytic | NTAVGKPVYCSAGNCPDIEKDAAIVVKSV |
| enzyme | IGTKTGIGAYVATDNARKEIVVSVRGSIN |
| [Fusarium | VRNWITNFDFGQKACDLVAGCGVHTGFLD |
| solani] | AWEEVAANIKAAVTAAKAANPTFKFVATG |
| | HSLGGAVATIAAAYLRKDGFPFDLYTYGS |
| | PRVGNDFFANFVTQQTGAEYRVTHGDDPV |
| | PRLPPIIFGYRHTSPEYWLDGGPLDKDYT |
| | VTEIKVCEGMPNVMCNGGTVGLDILAHIT |
| | YFQSMATGAPIAIPWKPHMSDEELEKKLT |
| | RYSELDQEFVKQMT |
| SEQ ID NO 8 | ATGAAGGGCTTCCTGCTGGCCAGCCTGGC |
| (DNA) PH2 | CGCCCTGGCCGTGGCCGCCCCCAGCAGCA |
| [Chaetomium | AGAAGCAGCGCGCCGCCCCCGTGACCGCC |
| thermophilum] | CAGCAGCTGAACAACTTCAAGCTGTACAT |
| | GCAGTGGAGCAGCGCCAGCCACTGCGCCA |
| | ACGAGGCCCCCATCGGCAGCGTGGTGACC |
| | TGCACCGACAACCAGTGCAGCATGTTCCA |
| | GAGCCACAACGCCACCGTGGCCGCCACCT |
| | TCATCGGCAGCATCCTGGACATGCGCGGC |
| | TTCCTGGGCATCGACGACGTGGACAAGAA |
| | CATCGTGCTGAGCTTCCGCGGCAGCACCA |
| | GCTGGCGCAACTGGATCGCCGACGCCATC |
| | TTCGTGCAGACCCCCTGCGACCTGACCCC |
| | CGGCTGCCTGGTGCACGCCGGCTTCTACG |
| | CCAGCTGGCTGGAGATCAAGAACAGCGTG |
| | ATCGACGCCGTGAAGGCCGCCAAGGCCGC |
| | CCACCCCAACTACAAGCTGGTGACCACCG |
| | GCCACAGCCTGGGCGCCGCCGTGGCCACC |
| | CTGGCCGCCGCCACCCTGCGCAAGGCCGG |
| | CATCCCCATCGAGCTGTACACCTACGGCA |
| | GCCCCCGCGTGGGCAACAAGGCCTTCGCC |
| | GAGTTCGTGACCAACCAGGCCGGCGGCGA |
| | GTACCGCCTGACCCACAGCGCCGACCCCA |
| | TCCCCCGCCTGCCCCCCATCATCTTCAAC |
| | TACCGCCACACCAGCCCCGAGTACTGGTT |
| | CGACGAGGGCGAGGACGGCGTGGTGACCG |
| | TGGACGAGTTCCAGATCTGCGAGGGCTAC |
| | GCCAACGTGAACTGCAACGCCGCCACCAG |
| | CGGCTTCAACATGGACCTGCACGGCTGGT |
| | ACTTCCAGAACCACCAGGGCTGCAGCCTG |
| | GGCTACACCCCCTGGCGCGCCGTGAAGGA |
| | GCGCGAGCTGAGCGACCCCGAGCTGGAGG |
| | CCCTGGTGAACCGCTTCGCCGAGATGGAC |
| | AAGGCCTACGTGGAGAACCTGAACCTGGA |
| | GGGCCTGGAGCCC |
| SEQ ID NO 9 | ATGGGCAAGCCCATGCGCTTCGCCGTGGG |
| (DNA) PH3 | CATCCTGGCCCTGCTGCTGGCCGCCTGCA |
| [Meiothermus | GCCAGCCCGCCACCGAGAGCACCAGCGCC |
| ruber] | AGCATCGTGGAGGACATGCGCCAGCTGGG |
| | CGTGGAGCCCGAGGCCATCGCCGCCTACA |
| | CCGAGGCCCTGCAGAACCTGGAGCAGGCC |
| | CGCCTGGCCCTGCAGAGCCTGGGCGCCGG |
| | CGACCTGCTGCTGGTGCAGAGCATCGCCC |
| | TGGGCAGCGTGGCCAACTACGACGCCTAC |
| | TACAACGCCCGCGCCAGCTACCCCCAGTT |
| | CGACTGGAGCCGCAACGGCTGCAGCGCCC |
| | CCGAGGGCCTGGGCCTGGGCTACCGCGAG |
| | ACCTTCCGCCCCGCCTGCAACGTGCACGA |
| | CTTCGGCTACGCCAACTTCCCCCGCTTCC |
| | CCAGCCTGTACAACGAGACCGGCCGCAAG |
| | CTGAGCGACGACAACTTCCTGGTGAACAT |
| | GAACCAGATCTGCCGCCCCCGCAGCTTCC |
| | TGAGCCGCAGCGCCTGCTACAGCGCCGCC |
| | TACGCCTACTACCTGGCCGTGCGCAGCGC |
| | CGGCTGGGCCTACTTCTACGAC |
| SEQ ID NO 10 | ATGCGCGTGGTGCTGCTGATGCTGGTGCT |
| (DNA) PH4 | GGTGCTGGCCGCCTGCGGCAGCCAGACCG |
| [Meiothermus | GCGCCCCCAACTGGACCCTGGAGCAGATC |
| silvanus] | GCCGCCCTGACCCCCGAGCAGATCGACGC |
| | CCTGAGCGAGGGCGAGCTGCAGAAGATCC |
| | AGACCATCCTGCAGCCCGAGTTCGCCCGC |
| | GTGGAGAGCCAGCTGATCCAGCTGCAGAG |
| | CCAGCTGGAGGCCCTGGTGGCCGCCCAGG |
| | ACAGCCGCTGGCCCAACTTCGACTACACC |
| | GTGTACCTGGCCACCGCCATCCCCTACGG |
| | CACCTTCTTCACCTACTACCGCACCTACA |
| | GCGGCCCCGACTGGAGCAACGACGGCTGC |
| | AGCTACAGCCCCGACAAGCCCCTGTTCCT |
| | GAACTTCAAGGACCCCTGCAACCACCACG |
| | ACTTCGGCTACCGC |

More particular said first enzyme is a lipolytic enzyme having a sequence identity of 100% to SEQ ID NO 1, SEQ ID NO 2, SEQ ID NO 3, SEQ ID NO 4 and/or SEQ ID NO 5, and preferably having a sequence identity of 100% with any of SEQ ID NO 1, SEQ ID NO 2 and/or SEQ ID NO 3, more preferably having a sequence identity of 100% with SEQ ID NO 2 and/or SEQ ID NO 3, and even more preferably having a sequence identity of 100% with SEQ ID NO 3.

In a particular embodiment the composition as disclosed herein provides that said first enzyme is a lipolytic enzyme with phospholipase activity from Chaetomium thermophilum is advantageously an enzyme with SEQ ID NO 1, or a close variant thereof. In a particular embodiment the composition as disclosed herein provides that said first enzyme is a lipolytic enzyme with phospholipase activity from Meiothermus ruber is advantageously an enzyme with SEQ ID NO 2, or a close variant thereof. In a particular embodiment the composition as disclosed herein provides that said first enzyme is a lipolytic enzyme with phospholipase activity from Meiothermus silvanus is advantageously an enzyme with SEQ ID NO 3, or a close variant thereof. In a particular embodiment the composition as disclosed herein provides that said first enzyme is a lipolytic enzyme with phospholipase activity from Streptomyces violaceoruber is advantageously an enzyme with SEQ ID NO 4, or a close variant thereof, more preferably Nagase 10P from Nagase. In a particular embodiment the composition as disclosed herein provides that said first enzyme is a lipolytic enzyme with phospholipase activity from Fusarium culmorum is advantageously an enzyme with SEQ ID NO 5, or a close variant thereof, more preferably Panamore® Golden from DSM.

In the context of the present invention a “close variant” as referred to herein is an enzyme that improves (the quality of) baked products as described above and that share a significant identity with SEQ ID NO 1 to 5. “Significant identity” in the context of the present invention refers to at least 85% identity, preferably at least 90% identity, preferably at least 91%, more preferably at least 92%, 93%, 94%, 95%, 96%, 97%, 98% A or even at least 99% with SEQ ID NO 1 to 5.

The relatedness between two amino acid sequences or between two nucleotide sequences is described by the parameter “identity”. For present purposes, the degree of identity between two amino acid sequences is determined as in WO 2010/0142 697 using the Needleman-Wunsch algorithm as implemented in the Needle program of the EMBOSS package, preferably version 3.0.0 or later. The optional parameters used are gap open penalty of 10, gap extension penalty of 0.5, and the EBLOSUM62 (EMBOSS version of BLOSUM62) substitution matrix. The output of Needle labelled “longest identity” (obtained using the −nobrief option) is used as the percent identity and is calculated as follows: (Identical Residues×100)/(Length of Alignment−Total Number of Gaps in Alignment).

In a particular embodiment the first enzyme is characterized by having an optimum phospholipase activity at a temperature equal or higher than 45° C.

In a particular embodiment the composition as disclosed herein provides that said first enzyme retains more than 50% of its phospholipase activity after being incubated for 30 minutes at 50° C.

The activity is measured by determining the phospholipase activity as indicated herein (using p-nitrophenyl phosphorylcholine as substrate).

In a particular embodiment the composition as disclosed herein provides that said second enzyme is chosen from a lipolytic enzyme with phospholipase and lipase activities from Thermomyces lanuginosus or a lipolytic enzyme with phospholipase and lipase activities from Fusarium solani.

In a particular embodiment the composition as disclosed herein provides that said second enzyme has a sequence identity of at least 85% with any of SEQ ID NO 6 and/or SEQ ID NO 7 (see Table A) or is chosen from Lipopan® Max from Novozymes or Veron® Hyperbake T from AB enzymes.

More particular said second enzyme is a lipolytic enzyme having a sequence identity of at least 85%, preferably at least 90%, more preferably at least 95% to SEQ ID NO 6 and/or SEQ ID NO 7 (see Table A).

More particular said second enzyme is a lipolytic enzyme having a sequence identity of 100% to SEQ ID NO 6 and/or SEQ ID NO 7.

In a particular embodiment the composition as disclosed herein provides that said second enzyme is a lipolytic enzyme with phospholipase activity from Thermomyces lanuginosus is advantageously an enzyme with SEQ ID NO 6, or a close variant thereof. In a particular embodiment the composition as disclosed herein provides that said first enzyme is a lipolytic enzyme with phospholipase activity from Fusarium solani is advantageously an enzyme with SEQ ID NO 7, or a close variant thereof.

Even more preferably the second enzyme is Lipopan® Max from Novozymes or Veron® Hyperbake T from AB enzymes.

In still preferred embodiments the composition as provided herein comprises an first enzyme having the amino acid sequence of SEQ ID NO 1, of SEQ ID NO 2, of SEQ ID NO 3, preferably of SEQ ID NO 2 or of SEQ ID NO 3, even more preferably of SEQ ID NO 3, or of close variants thereof and a second enzyme with a ratio phospholipase A1 activity/phospholipase A2 activity between 5000 and 60000, preferably between 10000 and 50000, more preferably between 15000 and 50000; and a ratio of phospholipase A1 activity/lipase activity equal or greater than 500.

In still preferred embodiments the composition as provided herein comprises a first enzyme having the amino acid sequence of SEQ ID NO 1, of SEQ ID NO 2, of SEQ ID NO 3, preferably of SEQ ID NO 2 or of SEQ ID NO 3, even more preferably of SEQ ID NO 3, or of close variants thereof and a second enzyme having the amino acid sequence of SEQ ID NO 6, of SEQ ID NO 7 or of close variant thereof, preferably a second enzyme having the sequence of SEQ ID NO 6 or a close variant thereof.

In even more preferred embodiments the composition as disclosed herein comprises a first enzyme having the amino acid sequence of SEQ ID NO 1 or of a close variant thereof and a second enzyme with a ratio phospholipase A1 activity/phospholipase A2 activity between 5000 and 60000, preferably between 10000 and 50000, more preferably between 15000 and 50000; and a ratio of phospholipase A1 activity/lipase activity equal or greater than 500, preferably a second enzyme having the amino acid sequence of SEQ ID NO 6, of SEQ ID NO 7 or of close variant thereof, more preferably a second enzyme having the sequence of SEQ ID NO 6 or a close variant thereof.

In even more preferred embodiments the composition as disclosed herein comprises a first enzyme having the amino acid sequence of SEQ ID NO 2 or of a close variant thereof and a second enzyme with a ratio phospholipase A1 activity/phospholipase A2 activity between 5000 and 60000, preferably between 10000 and 50000, more preferably between 15000 and 50000; and a ratio of phospholipase A1 activity/lipase activity equal or greater than 500, preferably a second enzyme having the amino acid sequence of SEQ ID NO 6, of SEQ ID NO 7 or of close variant thereof, more preferably a second enzyme having the sequence of SEQ ID NO 6 or a close variant thereof.

In even more preferred embodiments the composition as disclosed herein comprises a first enzyme having the amino acid sequence of SEQ ID NO 3 or of a close variant thereof and a second enzyme with a ratio phospholipase A1 activity/phospholipase A2 activity between 5000 and 60000, preferably between 10000 and 50000, more preferably between 15000 and 50000; and a ratio of phospholipase A1 activity/lipase activity equal or greater than 500, preferably a second enzyme having the amino acid sequence of SEQ ID NO 6, of SEQ ID NO 7 or of close variant thereof, more preferably a second enzyme having the sequence of SEQ ID NO 6 or a close variant thereof.

Furthermore, in a further aspect, the present invention relates to a bread improver comprising the composition as disclosed herein.

The composition of the present invention may advantageously be part of a bread improver or a patisserie mix or premix. “Bread improvers” (also referred to as “dough conditioners” or “dough improvers” or “improving agent” or “flour treatment agent”) are typically added to the dough in order to improve texture, volume, flavour and freshness of the baked product as well as to improve machinability and stability of the dough. Typically, a bread improver comprises or consists of: one or more enzymes (such as e.g. amylases (alpha-amylases, beta-amylases, glucoamylases, raw starch degrading amylases), xylanases (hemicellulases), cellulases, pectinases, proteases, pectate lyases, oxidases (peroxidases, glucose oxidase, pyranose oxidases, hexose oxydases, L-amino acid oxidases, carbohydrate oxidases, sulfurhydryl oxidases), lipoxygenases, dehydrogenases, laccases, transglutaminases, acyltransferases, protein disulfide isomerases), one or more oxidizing or reducing agents (such as e.g. ascorbic acid, glutathione, cysteine), one or more emulsifiers (such as e.g. diacetyl tartaric acid esters of monoglycerides (DATEM), sodium stearoyl lactylate (SSL), calcium stearoyl lactylate (CSL), glycerol monostearate (GMS), rhamnolipids, lecithins, sucroesters, bile salts), one or more lipid materials (such as e.g. margarine, butter, oil, shortening), one or more vitamins (such as e.g. pantothenic acid and vitamin E), one or more gums, and/or one or more sources of fibre (such as e.g. oat fibre). Cake (patisserie) mixes typically comprise all the ingredients of a cake recipe with the exception of water, fat (oil, butter, margarine) and eggs. Cake premixes are typically cake mixes where all or part of the flour and sugar has been removed.

In particular embodiments the composition comprises a first enzyme having a low phospholipase A1 activity/phospholipase A2 activity ratio and a second enzyme having a high phospholipase A1 activity/phospholipase A2 activity ratio as described above; and at least one, preferably two, additional ingredients chosen from the list of enzyme(s), oxidizing agent(s), reducing agent(s), emulsifier(s), lipid(s), vitamin(s), fibre(s). The inventors have found that it was particularly advantageous to include in the composition one or more enzyme(s) chosen from the group of amylases (alpha-amylases, beta-amylases, glucoamylases, raw starch degrading amylases), xylanases (hemicellulases), cellulases, pectinases, proteases, pectate lyases, oxidases (peroxidases, glucose oxidase, pyranose oxidases, hexose oxydases, L-amino acid oxidases, carbohydrate oxidases, sulfurhydryl oxidases), lipoxygenases, dehydrogenases, laccases, transglutaminases, acyltransferases, protein disulfide isomerases.

Furthermore, in a further aspect, the present invention relates to the use of a composition as disclosed herein in bakery applications. In the context of the present invention, bakery applications refer to applications related to both bread and patisserie products.

It has been found that the use of the compositions as provided herein allows the reduction or even the suppression of the use of undesired dough or batter ingredients such as emulsifiers.

In a particular embodiment the use of the composition as disclosed herein in bread improvers is provided.

In a particular embodiment the use of the composition as disclosed herein in bread or patisserie products, preferably cakes, bread, baguettes or rolls is provided.

Disclosed herein are also methods for preparing baked products wherein a first enzyme having a low phospholipase A1 activity/phospholipase A2 activity ratio and a second enzyme having a high phospholipase A1 activity/phospholipase A2 activity ratio are used in the preparation method.

Furthermore, in a further aspect, the present invention relates to a method for preparing a baked product, comprising the steps of adding to the dough or batter, prior to baking:
at least one first enzyme having a phospholipase A1 activity/phospholipase A2 activity ratio ranging between 0.01 and 100, preferably ranging between 0.01 and 80, more preferably ranging between 0.01 and 50, even more preferably ranging between 1 and 50, even more preferably ranging between 2 and 50, even more preferably ranging between, even more preferably between 0.01 and 25, even more preferably ranging between 1 and 25, and even more preferably ranging between 2 and 25; and;
at least one second enzyme having a phospholipase A1 activity/phospholipase A2 activity ratio ranging between 5000 and 60000, preferably between 10000 and 50000, more preferably between 15000 and 50000; and a phospholipase A1 activity/lipase activity ratio of 500 or more.

In said method the lipase activity is expressed in milliunits (LmU) that is defined as the amount of enzyme needed to release one nanomole (nmole) per minute of p-nitrophenol from p-nitrophenyl palmitate at 40° C. and pH 7.5, the phospholipase A1 activity is expressed in milliunits (PA1mU) that is defined as the amount of enzyme that hydrolyses one nanomole (nmole) per minute of fluorescent fatty acid substituted at the sn-1 position of N-((6-(2,4-DNP)Amino)Hexanoyl)-1-(BODIPY® FL C5)-2-Hexyl-Sn-Glycero-3-Phosphoethanolamine at 40° C. and pH7.4 and the phospholipase A2 activity is expressed in milliunits (PA2mU) per minute that is defined as the amount of enzyme that hydrolyses one nanomole (nmole) of fluorescent fatty acid substituted at the sn-2 position of 1-O-(6-BODIPY® 558/568-Aminohexyl)-2-BODIPY® FL C5-Sn-Glycero-3-Phosphocholine at 40° C. and pH 8.9 and the phospholipase activity is expressed in milliunits (PmU) that is defined as the amount of enzyme needed to release one nanomole (nmol) per minute of p-nitrophenol from p-nitrophenyl phosphorylcholine per minute at 50° C. and pH7.5.

In a particular embodiment of the method of the present invention the first enzyme is characterized by having an optimum phospholipase activity at a temperature equal or higher than 45° C.

It has been found that the compositions as disclosed herein are in particular suitable for use in the methods as disclosed herein.

In a particular embodiment the method as disclosed herein provides that said dough or batter comprises between 5000 and 100000 PmU/100 kg flour, preferably between 7000 and 50000 PmU/100 kg flour, more preferably between 10000 and 30000 PmU/100 kg flour of said first enzyme and between 10000 and 100000 LmU/100 kg flour, preferably between 20000 and 70000 LmU/100 kg flour of said second enzyme.

The method as disclosed herein advantageously allows to improve the batter or the dough tolerance and/or to improve the baked products properties, such as the volume or the freshness.

In a particular embodiment the method as disclosed herein provides that said dough or batter shows improved tolerance.

The inventors have found that the use of combination of a lipolytic enzyme and a particular phospholipase, in bakery applications, and in particular in the preparation of bread products has a synergistic effect on dough tolerance.

In the present context the dough tolerance refers to the capacity of a dough or a batter, preferably a bakery dough, to maintain its shape in stress conditions such a prolonged proofing time or mechanical shocks during or after proofing and to provide, after baking, a baked product with properties (e.g. volume) comparable to an baked product obtained with an unstressed dough or batter.

In a particular embodiment the method as disclosed herein provides that said baked product shows improved freshness.

In the present context freshness refers to a combination of texture parameters such as softness, moistness, cohesiveness, gumminess and resiliency. Loss of freshness is usually associated with staling. More particularly an improved freshness of a baked product corresponds to an improved softness and/or an improved moistness and/or an improved short bite when compared to a reference. These parameters may be advantageously measured by physical methods such as with a texturometer or by sensorial analysis conducted with a panel of expert judges.

Disclosed herein are also baked products comprising a first enzyme having a low phospholipase A1 activity/phospholipase A2 activity ratio and a second enzyme having a high phospholipase A1 activity/phospholipase A2 activity ratio.

Furthermore, in a further aspect, the present invention relates to a baked product prepared from a dough or batter comprising the composition as disclosed herein.

In the context of the present invention a baked product is a bakery or patisserie product known in the art, such as for instance those selected from the group comprising bread, soft rolls, bagels, donuts, Danish pastry, hamburger rolls, pizza, pita bread, ciabatta, sponge cakes, cream cakes, pound cakes, muffins, cupcakes, steamed cakes, waffles, brownies, cake donuts, yeast raised donuts, baguettes, rolls, crackers, biscuits, cookies, pie crusts, rusks and other baked products. More preferably the present invention refers to bread, baguettes and rolls.

A further object of the present invention relates to the use of compositions, bread improvers, patisserie mixes and/or patisserie premixes to prepare baked products.

### Examples

### Example 1: Enzymatic Activities Determination

### Lipase:

The lipase activity is measured using p-nitrophenyl palmitate (pNPP) as substrate. The release of yellow p-nitrophenol due to hydrolysis of p-nitrophenyl palmitate by lipase is measured by spectrophotometry at 414 nm. One lipase milliunit (LmU) is defined as the amount of enzyme needed to release one nanomole (nmole) per minute of p-nitrophenol from p-nitrophenyl palmitate at 40° C. and pH 7.5. To perform the test, 120 μl of 1 mM pNPP solution (dissolved in 0.05 M Na-phosphate buffer at pH 7.5 containing 0.69 M acetone and 0.0049 M Triton X-100) are mixed with 60 μl of enzyme sample and incubated at 40° C. for 30 min. The absorbance is measured at 414 nm against a substrate blank in 96-wells microplates.

The activity is expressed as: LmU/ml=(((Abs enzyme−Abs blank)/30)×0.18))/(13380×0.06))×sample dilution×1000000

[30=reaction time in minutes; 0.18=reaction volume in ml; 13380=molar extinction coefficient at 414 nm (M⁻¹ cm⁻¹); 0.06=enzyme sample volume in ml; 1000000 to convert in LmU/ml]

### Phospholipase:

The phospholipase activity is measured using p-nitrophenyl phosphorylcholine (pNPPC) as substrate. The release of yellow p-nitrophenol due to hydrolysis of p-nitrophenyl phosphorylcholine by phospholipase is measured by spectrophotometry at 414 nm. One phospholipase milliunit (PmU) is defined as the amount of enzyme needed to release 1 nmol of p-nitrophenol per minute at 50° C. and pH7.5. To perform the test, 120 μl of 0.02 M pNPPC solution (dissolved in 0.05 M Na-phosphate buffer at pH 7.5 containing 0.69 M acetone and 0.0049 M Triton X-100) are mixed with 60 μl of enzyme sample and incubated at 50° C. for 30 min. Afterwards, 720 μl of 1M Na₂CO₃ are added to stop the reaction. The absorbance is measured at 414 nm against a substrate blank in 96-wells microplates.

The activity is expressed as: PmU/ml=(((Abs enzyme−Abs blank)/30)×0.9))/(13380×0.06))×sample dilution×1000000

[30=reaction time in minutes; 0.9=reaction volume in ml; 13380=molar extinction coefficient at 414 nm (M⁻¹ cm⁻¹); 0.06=enzyme sample volume in ml; 1000000 to convert in PmU/ml]

### Phospholipase A1:

The phospholipase A1 (PLA1) activity is measured using a lipid mix of 16.5 μM dioleoylphosphatidylcholine, 16.5 μM dioleoylphosphatidylglycerol and 3.3 μM dye labelled N-((6-(2,4-DNP)Amino)Hexanoyl)-1-(BODIPY® FL C5)-2-Hexyl-Sn-Glycero-3-Phosphoethanolamine (PED-A1) as substrate (obtained in the EnzChek Phospholipase A1 assay kit—ThermoFisher Scientific). The PED-A1 is specific for PLA1 and is a dye-labelled glycerophosphoethanolamine with BODIPY® FL dye-labelled acyl chain at the sn-1 position and dinitrophenyl quencher-modified head group. One phospholipase A1 milliunit (PM mU) is defined as the amount of enzyme needed to release one nanomole (nmole) per minute of fluorescent fatty acid substituted at the sn-1 position of PED-A1 at 40° C. and pH7.4. The test is performed in 96-wells microplates using a total volume of 100 μl per well. Different lipid solutions were prepared in reaction buffer containing 250 mM Tris-HCl, 0.7 M NaCl and 10 mM CaCl₂ at pH 7.4. Samples and controls are mixed with the lipid-mix at a ratio of 1:1 (50 μl sample/control and 50 μl lipid mix). The enzymatic reaction is performed at 40° C. during 30 min. A calibration curve is established by use of different concentrations of phospholipase A1 provided in the kit (Lecitase ultra). The fluorescence measurement is performed with excitation at 480 nm and with emission at 515 nm.

The activity is expressed as: PA1mU/ml=(((fluorescence intensity enzyme−fluorescence intensity blank)−intercept value)/slope value of calibration curve)×sample dilution×1000 [1000 to convert in PA1mU/ml]

### Phospholipase A2:

The phospholipase A2 (PLA2) activity is measured using a lipid mix of 16.5 μM dioleoylphosphatidylcholine, 16.5 μM dioleoylphosphatidylglycerol and 1.7 μM dye labelled 1-O-(6-BODIPY® 558/568-Aminohexyl)-2-BODIPY® FL C5-Sn-Glycero-3-Phosphocholine (Red/Green BODIPY® PC-A2) as substrate (obtained in the EnzChek Phospholipase A2 assay kit—ThermoFisher Scientific). The Red/Green BODIPY® PC-A2 substrate is selective for PLA2 and provides sensitive and continuous rapid real-time monitoring of PLA2 enzyme activities. One phospholipase A2 milliunit (PA2mU) is defined as the amount of enzyme needed to release one nanomole (nmole) per minute of fluorescent fatty acid substituted at the sn-2 position of Red/Green BODIPY® PC-A2 at 40° C. and pH 8.9. The test is performed 96-wells micro plates using a total volume of 100 μl per well. Different lipid solutions were prepared in reaction buffer containing 250 mM Tris-HCl, 500 mM NaCl and 5 mM CaCl₂ at pH 8.9. Samples and controls are mixed with lipid-mix at a ratio of 1:1 (50 μl sample/control and 50 μl lipid mix). The enzymatic reaction is performed at 40° C. during 10 min. A calibration curve is established by use of different concentrations of phospholipase A2 from honey bee venom provided in the kit. The fluorescence measurement is performed with excitation at 480 nm and with emission at 515 nm.

The activity is expressed as: PA2mU/ml=(((fluorescence intensity enzyme−fluorescence intensity blank)−intercept value)/slope value of calibration curve)×sample dilution×1000 [1000=PA2mU/ml]

### Example 2: Enzymes

The following enzymes were used in the following examples.
Lipolytic enzyme with phospholipase activity from Chaetomium thermophilum (PH2) having the SEQ NO 1 amino acid sequence.
Lipolytic enzyme with phospholipase activity from Meiothermus ruber (PH3) having the SEQ NO 2 amino acid sequence.
Lipolytic enzyme with phospholipase activity from Meiothermus silvanus (PH4) having the SEQ NO 3 amino acid sequence.
Lipopan® Max (lipase from Thermomyces lanuginosus; Novozymes) (LIM)
Nagase 10P (phospholipase A2 from; Streptomyces violaceoruber; Nagase) (NAG)
Panamore® Golden (lipase from Fusarium culmorum; DSM) (PAN)
Lecitase Ultra (protein-engineered carboxylic ester hydrolase from Thermomyces lanuginosus; Novozymes) (LEC)
Bakezyme® PH 800 BG (lipase from Fusarium oxysporum; DSM) (BAK)
Veron® Hyperbake-T (lipase from Fusarium solani; AB enzymes) (HYP)

The PH2, PH3 and PH4 enzymes have been obtained by cloning and expressing the corresponding genes as described hereafter. The other enzymes were obtained from their respective suppliers.

### Cloning of the PH2, PH3 and PH4 Genes

Based on the identified sequences of putative lipolytic enzymes genes the DNA sequence coding for the enzymes have been synthesized in order to be expressed into the pET22b plasmid by using the pelB leader sequence and following standard protocols.

The DNA sequences corresponding to PH2, PH3 and PH4 are shown in Table A and are respectively SEQ ID NO 8, SEQ ID NO 9 and SEQ ID NO 10 (see table A).

The complete synthesized DNA fragments were subcloned into a pUC19 derivative plasmid. These plasmids were used to transform E. coli DH5α® ultracompetent cells. Purified plasmid preparations made with the Pure Yield Midiprep System (Promega) were digested by using appropriate restriction enzymes to isolate the DNA fragment containing the lipolytic enzymes coding sequences. Those fragments were subcloned into the pET 22b(+) cloning vector (Novagen) and the resulting recombinant plasmids were transformed in E. coli BL21 (DE3) cells (Agilent Technologies). Purified plasmid preparations made with the Pure Yield Midiprep System (Promega) were sequenced by using a AB13700 DNA sequencer (Applied Biosystems). Sequencing of the inserted fragments was carried out using the universal primers T7 promoter and T7 terminator as well as primers corresponding to internal DNA sequences. The sequences obtained were identical to the expected sequences.

### Cultures of the Recombinant Strains and Production of PH2, PH3 and PH4

15 ml of a 5 hours preculture (37° C.) of the E. coli BL21 (DE3) cells carrying the lipolytic enzyme genes were centrifuged at 10000 g for 1 minute and the pellet was resuspended in 500 ml Terrific broth (12 g/l Bacto tryptone (Difco), 24 g/l yeast extract (Difco), 4 ml/l glycerol, 12.54 g/l K₂HPO₄, 2.31 g/l KH₂PO₄) containing 200 μg/ml ampicillin in a 2 liters shake flask. The cultures were incubated at 37° C. and 250 rpm until an absorbance at 550 nm of between 3 & 4 was reached whereupon the expression of the enzyme was induced with 1 mM isopropyl-1-thio-8-galactopyranoside.

### Recovery of PH2, PH3 and PH4

After 15 hours incubation at 37° C. the cells were harvested by centrifugation at 18000 g for 30 minutes at 4° C., resuspended in 50 mM BICINE containing 10 mM NaCl, disrupted in a prechilled cell disrupter (Panda 2K, Niro Soavi, GEA Process Engineering Division) at 1500 bars and centrifuged at 40,000 g for 30 minutes. Chromosomal DNA was removed from the crude cell lysates by treatment with 0.2% protamine sulfate (Calbiochem) and centrifugation at 40,000 g for 30 minutes. 25 units of benzonase (Merck, Darmstadt, Germany) were then added to the solution. After such a treatment the lipolytic enzyme preparations have been clarified by an end filtration on a Millipore POD system with a range of cut-off from 0.05 to 1 μm then concentrated by ultrafiltration on a cross flow filtration system (Satocon-Sartorius) with a cut-off of 5 kDa. The concentrated enzyme solutions was filtered on a sterile filtration system, including end filtration of 0.8 and 0.22 μm (absolute filter).

The lipases and phospholipases activities of the enzymes have been determined using the protocols of example 1. The results are shown in table 1.

TABLE 1

| | PL | PLA1 | PLA2 | Lipase (L) | | |
|---|---|---|---|---|---|---|
| | activity | activity | activity | activity | | |
| En- | (PmU/ | (PA1mU/ | (PA2mU/ | (LmU/ | PLA1/ | |
| zyme | ml or g) | ml or g) | ml or g) | ml or g) | PLA2 | PLA1/L |
| PH2 | 140 | 13 | 450 | 11 | 0.03 | 1 |
| PH3 | 27 | 111406 | 31634 | 16 | 3.52 | 6963 |
| PH4 | 33 | 94400 | 14732 | 15 | 6.41 | 6293 |
| LIM | 0.4 | 94410920 | 4512 | 10913 | 20920 | 8650 |
| NAG | 148 | 41307 | 13520 | 27.47 | 3.06 | 1504 |
| PAN | 15 | 171226 | 3652 | 244644 | 47 | 0.70 |
| LEC | 144 | 54983000 | 204111 | 169200 | 269 | 325.0 |
| BAK | 64 | 33722000 | 14038 | 372945 | 2402 | 90.4 |
| HYP | 85 | 42339770 | 1000 | 60174 | 42340 | 703.6 |

The activity of the enzymes in function of the temperature has been determined using the phospholipase assay of example 1 except for the temperature of the test. Results are presented in Table 2.

TABLE 2

Activity (% of maximum activity)

| Enzyme | 30° C. | 40° C. | 50° C. | 60° C. |
|---|---|---|---|---|
| PH2 | 23 | 84 | 100 | 34 |
| PH3 | 40 | 75 | 100 | 92 |
| PH4 | 50 | 67 | 93 | 100 |
| NAG | 2 | 4 | 37 | 100 |
| PAN | 33 | 58 | 83 | 100 |

The stability of the enzymes has been determined by preincubating a sample of the enzyme at 50° C. for 30, 60 and 240 minutes before performing the phospholipase assay as in example 1. Results are presented in Table 3.

TABLE 3

| | residual activity (%) | | |
|---|---|---|---|
| Enzyme | 30 min | 60 min | 240 min |
| PH2 | 106 | 102 | 64 |
| PH3 | 59 | 52 | 41 |
| PH4 | 87 | 86 | 95 |
| NAG | 36 | 26 | 25 |
| PAN | 33 | 0 | 0 |

### Example 3: Crusty Rolls

Effect of combination of lipolytic enzymes was tested in crusty rolls making. Crusty rolls were prepared using the dough compositions of Table 4.

TABLE 4

| Ingredients (grams) | |
|---|---|
| Wheat Flour (Crousti; Belgium)) | 2100 |
| Water | 1260 |
| Fresh Yeast (Bruggeman, Belgium) | 126 |
| Sodium Chloride | 42 |
| Improver* | 21 |
| Enzymes | |
| Enzyme 1 (PmU) | 315 |
| Enzyme 2 (LmU) | 690 |

*contains ascorbic acid and enzymes (alpha amylase, xylanase)

The ingredients were mixed for 2 min at low and 5 min at high speed in an Eberhardt N24 mixer. The final dough temperature as well as the resting and proofing temperatures were 25° C. After resting for 15 min at 25° C., the dough was reworked manually and rested for another 10 min. Afterwards, 2 kg dough pieces were made up and proofed for 10 min. The 2-kg dough pieces were divided and made up using the Rotamat. 50 gr. round dough pieces were obtained. After another 5 min resting time, the dough pieces were cut by pressing and 50% of the dough pieces were submitted to a final proofing stage at 35° C. for 120 min and 50% of the dough pieces were submitted to a shock test (the tray containing the doughs is dropped on the shelf from a height of 10 cm) and submitted to a final proofing stage at 35° C. for 120 min. The dough pieces were baked at 230° C. in a MIWE Roll-In oven with steam (Michael Wenz-Arnstein-Germany). The volume of 6 rolls was measured using the commonly used rapeseed displacement method.

The results are shown in Table 5.

TABLE 5

| | | Rolls volume (ml per 6) | | | | | | | | | | | |
|---|---|---|---|---|---|---|---|---|---|---|---|---|---|
| | | 120 min proofing | | | | | 120 min proofing + Shock test | | | | | loss | loss |
| | | Enzyme(s) in test | | | | | Enzyme(s) in test | | | | | 1 + 2 | ½ |
| Enzyme 1 | Enzyme 2 | none | 1 | 2 | 1 + 2 | ½* | none | 1 | 2 | 1 + 2 | ½* | (%)** | (%)** |
| PH2 | LIM | 1900 | 2025 | 2225 | 2300 | 2350 | 1400 | 1475 | 1675 | 1875 | 1750 | −18 | −26 |
| PH3 | LIM | 1875 | 2025 | 2125 | 2325 | 2275 | 1325 | 1300 | 1550 | 1850 | 1525 | −20 | −33 |
| PH4 | LIM | 1975 | 2050 | 2220 | 2425 | 2295 | 1325 | 1500 | 1600 | 1875 | 1775 | −23 | −23 |
| NAG | LIM | 2100 | 2250 | 2275 | 2375 | 2425 | 1325 | 1425 | 1675 | 1775 | 1775 | −25 | −27 |
| PAN | LIM | 2050 | 2100 | 2175 | 2325 | 2225 | 1225 | 1325 | 1500 | 1650 | 1600 | −29 | −28 |
| LEC | LIM | 2050 | — | 2175 | 1925 | — | 1225 | — | 1500 | 1325 | — | — | — |
| HYP | LIM | 2000 | — | 2275 | 1975 | — | 1225 | — | 1475 | 1400 | — | — | — |
| BAK | LIM | 2000 | — | 2275 | 1875 | — | 1225 | — | 1475 | 1350 | — | — | — |

*calculated as the sum of the volume increases (compared to control) given by each enzyme alone
**calculated as percentage of volume loss given by the shock test

The results show that the use of an enzyme 1 that corresponds to the first enzyme of a composition according to the invention and of an enzyme 2 that corresponds to the second enzyme of a composition according to the invention (“1+2”) gives a better volume improvement and a better dough tolerance than the reference (“none”) or than when the enzymes are used alone (“1” or “2”). Furthermore this effect is synergistic as the enzyme combinations gives results that are higher than the sum of the results obtained with the enzymes used individually (“½”).

### Example 4: Crusty Rolls

Effect of combination of lipolytic enzymes was tested in crusty rolls making.

Crusty rolls were prepared and evaluated as in example 3. Enzymes were added to the dough according to the scheme of Table 6. The enzymes dosages were the following: PH3 315 PmU/dough; LIM 690 LmU/dough; HYP 1200 LmU/dough; LEC 1400 LmU/dough.

TABLE 6

| | | Rolls volume (ml per 6) | | | | | |
|---|---|---|---|---|---|---|---|
| | | | | | 120 min | | |
| | | 120 min proofing | | | proofing + Shock test | | |
| | | Enzyme(s) in test | | | Enzyme(s) in test | | |
| Enzyme 1 | Enzyme 2 | — | 2 | 1 + 2 | — | 2 | 1 + 2 |
| PH3 | LIM | 1850 | 2150 | 2175 | 1350 | 1550 | 1675 |
| PH3 | HYP | 1850 | 2250 | 2375 | 1350 | 1775 | 1900 |
| PH3 | LEC | 1850 | 2125 | 2150 | 1350 | 1775 | 1775 |

The results show that the use of an enzyme 1 (PH3) that corresponds to the first enzyme of a composition according to the invention and of an enzyme 2 (LIM or HYP) that corresponds to the second enzyme of a composition according to the invention gives a better volume improvement and a better dough tolerance than the reference or than when the enzymes are used alone.

### Example 5: Porcine Pancreatic Lipase

The enzymatic activities of example 1 of the phospholipase A2 from porcine pancreas (PPL; obtained from Sigma, ref. P6534-10MG) have been determined.

TABLE 7

| | | PLA1 | PLA2 | | | |
|---|---|---|---|---|---|---|
| | PL | activity | activity | Lipase (L) | | |
| | activity | (PA1mU/ | (PA2mU/ | activity | PLA1/ | |
| | (PmU/ml) | ml) | ml) | (LmU/ml) | PLA2 | PLA1/L |
| PPL | 0.37 | 27540 | 57087 | 0.74 | 0.48 | 37216 |

The protein content of the purified enzyme is 3.66 mg/ml

Crusty rolls were prepared and evaluated as in example 3. The enzymes doses were respectively: LIM: 690 LmU/dough; PH4: 703 PmU/dough; PPLa: 0.44 mg/dough; PPLb: 1.76 mg/dough.

The results are presented in table 8.

TABLE 8

| | Rolls volume (ml per 6) | | |
|---|---|---|---|
| | | 120 min proofing + | Volume |
| Enzyme(s) | 120 min proofing | shock test | loss (%) |
| none | 2225 | 1550 | −30 |
| LIM | 2300 | 1800 | −22 |
| PH4 | 2225 | 1650 | −26 |
| PPLa | 2225 | 1625 | −27 |
| PPLb | 2225 | 1625 | −27 |
| LIM + PH4* | 2325 | 2000 | −14 |
| LIM + PPLa* | 2400 | 1950 | −19 |
| LIM + PPLb* | 2375 | 1925 | −19 |
| LIM/PH4** | 2300 | 1900 | −17 |
| LIM/PPLa** | 2300 | 1875 | −18 |
| LIM/PPLb** | 2300 | 1875 | −18 |

*enzymes added simultaneously in the test
**volumes calculated by summing up the volumes increases given by each individual enzyme.

These results show that the porcine pancreatic phospholipase A2 has no synergistic effect on dough tolerance and that increasing the dose of the enzyme has no effect.

### Example 6: Emulsifier Replacement

Effect of combination of lipolytic enzymes was tested for the replacement of emulsifiers in piccolos making (overnight method). Piccolos were prepared using the dough compositions of Table 9.

TABLE 9

| Ingredients (grams) | |
|---|---|
| Wheat Flour (Crousti; Belgium)) | 1100 |
| Water | 638 |
| Fresh Yeast (Bruggeman, Belgium) | 44 |
| Sodium Chloride | 22 |
| Improver* | 33 |
| Additional ingredient | According to table 10 |

*contains ascorbic acid and enzymes (alpha amylase, xylanase)

The ingredients were mixed for 2 min at low and 5 min at high speed in an Eberhardt N24 mixer. The final dough temperature as well as the resting and proofing temperatures were 25° C. After resting for 5 min at 25° C., the dough was reworked manually and rested for another 5 min. Afterwards the doughs were divided and made up using a Rotamat. 70 gr. dough pieces were obtained. The dough pieces were stored overnight at about 2° C. in a Koma proofbox. The temperature of the proofbox was progressively increased to 25° C. in a 6 hours period. After a final proof of 30 minutes at 25° C. and 95% humidity, the dough pieces were cut once with a knife and baked at 230° C. in a MIWE Roll-In oven with steam (Michael Wenz-Arnstein-Germany) for 18 minutes. The volume of 6 piccolos was measured using the commonly used rapeseed displacement method.

The results are shown in Table 10.

TABLE 10

| | Piccolos volume |
|---|---|
| Additional ingredient (amount/dough) | (ml/per 6) |
| DATEM (diacetyl tartaric acid ester of | 2625 |
| monoglycerides- MD HP20; Puratos; Belgium): 3.3 g | |
| DATEM (diacetyl tartaric acid ester of | 2750 |
| monoglycerides- MD HP20; Puratos; Belgium): 5.5 g | |
| LIM: 360 LmU | 2625 |
| LIM: 600 LmU | 2650 |
| LIM: 360 LmU + PH3: 165 PmU | 2825 |
| LIM: 360 LmU + PH4: 165 PmU | 2850 |
| LIM: 360 LmU + PH4: 407 PmU | 2900 |

The results show that the combination of enzymes according to the invention allows the full replacement of emulsifiers in the dough recipe.

1. A composition comprising:
at least one first enzyme having a phospholipase A1 activity/phospholipase A2 activity ratio ranging between 0.01 and 100, preferably ranging between 0.01 and 80, more preferably ranging between 0.01 and 50, more preferably ranging between 1 and 50, even more preferably ranging between 2 and 50, even more preferably between 0.01 and 25, even more preferably ranging between 1 and 25, and even more preferably ranging between 2 and 25; wherein said first enzyme is characterized by having an optimum phospholipase activity at a temperature equal or higher than 45° C.; and wherein said first enzyme has a sequence identity of at least 85% with any of SEQ ID NO 1, SEQ ID NO 2 and/or SEQ ID NO 3, more preferably having a sequence identity of at least 85% with any of SEQ ID NO 2 and/or SEQ ID NO 3, and even more preferably having a sequence identity of at least 85% with SEQ ID NO 3; and;
at least one second enzyme having a phospholipase A1 activity/phospholipase A2 activity ratio ranging between 5000 and 60000, preferably between 10000 and 50000, more preferably between 15000 and 50000; and a phospholipase A1 activity/lipase activity ratio of 500 or more; wherein said second enzyme has a sequence identity of at least 85% with any of SEQ ID NO 6 and/or SEQ ID NO 7.

2. The composition according to claim 1, wherein said first enzyme is chosen from a lipolytic enzyme with phospholipase activity from Chaetomium thermophilum, a lipolytic enzyme with phospholipase activity from Meiothermus ruber, and/or a lipolytic enzyme with phospholipase activity from Meiothermus silvanus.

3. The composition according to claim 1, wherein said second enzyme is chosen from a lipolytic enzyme with phospholipase and lipase activities from Thermomyces lanuginosus or a lipolytic enzyme with phospholipase and lipase activities from Fusarium solani.

4. Use of a composition according to claim 1 in bakery applications.

5. Use according to claim 4 in bread improvers.

6. Use according to claim 4 in bread or patisserie products, preferably cakes, bread, baguettes or rolls.

7. Bread improver comprising the composition according to claim 1.

8. A method for preparing a baked product, comprising the steps of adding to the dough or batter, prior to baking:
at least one first enzyme having a phospholipase A1 activity/phospholipase A2 activity ratio ranging between 0.01 and 100, preferably ranging between 0.01 and 80, more preferably ranging between 0.01 and 50, even more preferably ranging between 1 and 50, even more preferably ranging between 2 and 50, even more preferably ranging between 0.01 and 25, even more preferably ranging between 1 and 25, and even more preferably ranging between 2 and 25; wherein said first enzyme is characterized by having an optimum phospholipase activity at a temperature equal or higher than 45° C.; and wherein said first enzyme has a sequence identity of at least 85% with any of SEQ ID NO 1, SEQ ID NO 2 and/or SEQ ID NO 3, more preferably having a sequence identity of at least 85% with any of SEQ ID NO 2 and/or SEQ ID NO 3, and even more preferably having a sequence identity of at least 85% with SEQ ID NO 3; and;
at least one second enzyme having a phospholipase A1 activity/phospholipase A2 activity ratio ranging between 5000 and 60000, preferably between 10000 and 50000, more preferably between 15000 and 50000; and a phospholipase A1 activity/lipase activity ratio of 500 or more; wherein said second enzyme has a sequence identity of at least 85% with any of SEQ ID NO 6 and/or SEQ ID NO 7.

9. The method according to claim 8, wherein said dough or batter comprises between 5000 and 100000 PmU/100 kg flour, preferably between 7000 and 50000 PmU/100 kg flour, more preferably between 10000 and 30000 PmU/100 kg flour of said first enzyme and between 10000 and 100000 LmU/100 kg flour, preferably between 20000 and 70000 LmU/100 kg flour of said second enzyme.

10. The method according to claim 8, wherein said dough or batter shows improved tolerance.

11. The method according claim 8, wherein said baked product shows improved freshness.

12. Baked product prepared from a dough or batter comprising the composition according to claim 1.


SEQUENCE LISTING (10 sequences)

SEQ ID NO: 1 - protein, 333 aa, Chaetomium thermophilum
MKGFLLASLAALAVAAPSSKKQRAAPVTAQQLNNFKLYMQWSSASHCANEAPIGSVVTCTDNQCSMFQSHNATVAATFIGSILDMRGFLGIDDVDKNIVLSFRGSTSWRNWIADAIFVQTPCDLTPGCLVHAGFYASWLEIKNSVIDAVKAAKAAHPNYKLVTTGHSLGAAVATLAAATLRKAGIPIELYTYGSPRVGNKAFAEFVTNQAGGEYRLTHSADPIPRLPPIIFNYRHTSPEYWFDEGEDGVVTVDEFQICEGYANVNCNAATSGFNMDLHGWYFQNHQGCSLGYTPWRAVKERELSDPELEALVNRFAEMDKAYVENLNLEGLEP

SEQ ID NO: 2 - protein, 191 aa, Meiothermus ruber
MGKPMRFAVGILALLLAACSQPATESTSASIVEDMRQLGVEPEAIAAYTEALQNLEQARLALQSLGAGDLLLVQSIALGSVANYDAYYNARASYPQFDWSRNGCSAPEGLGLGYRETFRPACNVHDFGYANFPRFPSLYNETGRKLSDDNFLVNMNQICRPRSFLSRSACYSAAYAYYLAVRSAGWAYFYD

SEQ ID NO: 3 - protein, 140 aa, Meiothermus silvanus
MRVVLLMLVLVLAACGSQTGAPNWTLEQIAALTPEQIDALSEGELQKIQTILQPEFARVESQLIQLQSQLEALVAAQDSRWPNFDYTVYLATAIPYGTFFTYYRTYSGPDWSNDGCSYSPDKPLFLNFKDPCNHHDFGYR

SEQ ID NO: 4 - protein, 150 aa, Streptomyces violaceoruber
MRTTTRTRTTLAAVGAALALGVAAAPPQAAPADKPQVLASFTQTSASSQNAWLAANRNQSAWAAYEFDWSTDLCSQAPDNPFGFPFNTACARHDFGYRNYKAAGSFDANKSRIDSAFYEDMKRVCTGYTGEKNTACNSTAWTYYQAVKIL

SEQ ID NO: 5 - protein, 436 aa, Fusarium culmorum
MVLDRLLFLLSLWLGFVGATQAALSEPIPPSKDPWYTAPPGFENAEPGTVFRVRPAPGNLTSVIGNCSASYNILYRTTDSHFKPTWAVTTLLVPKLGPESLAQQKYQQSALLSFQVPYDSPDVDASPSNAMYDASDFFSNYYGAALGEGIFVSVPDYEGPLAAFTAGLISGYATLDSIRAILSLGLGFNTIDTPSVALWGYSGGAFATEWASELAVQYAPELVAGPVIGAVMGAPLPNITSCMRDVNGGPKSGLVVNILLGLTGQYPDVRKHLVSKLNDDGQYNKAGFLAAEGFTISEALSTFSGNINKYFQKGTDILSDPKITALINREGVLGYHGTPRWPMFIYQAISDEVTPIAATDAVVERYCSVGADVHFERNTLGSHDEEAGNSYDAAFQWLLDIFSGQRDTKGCVIKDVTRDVTGDVTGDVTRDVTREL

SEQ ID NO: 6 - protein, 291 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 7 - protein, 333 aa, Fusarium solani
MMLILSILSIIAFTAAGPVPSVDENTRVLEHRAVTVTTQDLSNFRFYLQHADAAYCNFNTAVGKPVYCSAGNCPDIEKDAAIVVKSVIGTKTGIGAYVATDNARKEIVVSVRGSINVRNWITNFDFGQKACDLVAGCGVHTGFLDAWEEVAANIKAAVTAAKAANPTFKFVATGHSLGGAVATIAAAYLRKDGFPFDLYTYGSPRVGNDFFANFVTQQTGAEYRVTHGDDPVPRLPPIIFGYRHTSPEYWLDGGPLDKDYTVTEIKVCEGMPNVMCNGGTVGLDILAHITYFQSMATGAPIAIPWKPHMSDEELEKKLTRYSELDQEFVKQMT

SEQ ID NO: 8 - DNA, 999 bp, Chaetomium thermophilum
atgaagggcttcctgctggccagcctggccgccctggccgtggccgcccccagcagcaagaagcagcgcgccgcccccgtgaccgcccagcagctgaacaacttcaagctgtacatgcagtggagcagcgccagccactgcgccaacgaggcccccatcggcagcgtggtgacctgcaccgacaaccagtgcagcatgttccagagccacaacgccaccgtggccgccaccttcatcggcagcatcctggacatgcgcggcttcctgggcatcgacgacgtggacaagaacatcgtgctgagcttccgcggcagcaccagctggcgcaactggatcgccgacgccatcttcgtgcagaccccctgcgacctgacccccggctgcctggtgcacgccggcttctacgccagctggctggagatcaagaacagcgtgatcgacgccgtgaaggccgccaaggccgcccaccccaactacaagctggtgaccaccggccacagcctgggcgccgccgtggccaccctggccgccgccaccctgcgcaaggccggcatccccatcgagctgtacacctacggcagcccccgcgtgggcaacaaggccttcgccgagttcgtgaccaaccaggccggcggcgagtaccgcctgacccacagcgccgaccccatcccccgcctgccccccatcatcttcaactaccgccacaccagccccgagtactggttcgacgagggcgaggacggcgtggtgaccgtggacgagttccagatctgcgagggctacgccaacgtgaactgcaacgccgccaccagcggcttcaacatggacctgcacggctggtacttccagaaccaccagggctgcagcctgggctacaccccctggcgcgccgtgaaggagcgcgagctgagcgaccccgagctggaggccctggtgaaccgcttcgccgagatggacaaggcctacgtggagaacctgaacctggagggcctggagccc

SEQ ID NO: 9 - DNA, 573 bp, Meiothermus ruber
atgggcaagcccatgcgcttcgccgtgggcatcctggccctgctgctggccgcctgcagccagcccgccaccgagagcaccagcgccagcatcgtggaggacatgcgccagctgggcgtggagcccgaggccatcgccgcctacaccgaggccctgcagaacctggagcaggcccgcctggccctgcagagcctgggcgccggcgacctgctgctggtgcagagcatcgccctgggcagcgtggccaactacgacgcctactacaacgcccgcgccagctacccccagttcgactggagccgcaacggctgcagcgcccccgagggcctgggcctgggctaccgcgagaccttccgccccgcctgcaacgtgcacgacttcggctacgccaacttcccccgcttccccagcctgtacaacgagaccggccgcaagctgagcgacgacaacttcctggtgaacatgaaccagatctgccgcccccgcagcttcctgagccgcagcgcctgctacagcgccgcctacgcctactacctggccgtgcgcagcgccggctgggcctacttctacgac

SEQ ID NO: 10 - DNA, 420 bp, Meiothermus silvanus
atgcgcgtggtgctgctgatgctggtgctggtgctggccgcctgcggcagccagaccggcgcccccaactggaccctggagcagatcgccgccctgacccccgagcagatcgacgccctgagcgagggcgagctgcagaagatccagaccatcctgcagcccgagttcgcccgcgtggagagccagctgatccagctgcagagccagctggaggccctggtggccgcccaggacagccgctggcccaacttcgactacaccgtgtacctggccaccgccatcccctacggcaccttcttcacctactaccgcacctacagcggccccgactggagcaacgacggctgcagctacagccccgacaagcccctgttcctgaacttcaaggacccctgcaaccaccacgacttcggctaccgc